(19) United States
(12) Patent Application Publication
(10) Pub. No.: US 2023/0389824 A1
(43) Pub. Date: Dec. 7, 2023

(54) ESTIMATING GAIT EVENT TIMES & GROUND CONTACT TIME AT WRIST

(71) Applicant: Apple Inc., Cupertino, CA (US)

(72) Inventors: Allison L. Gilmore, Redwood City, CA (US); Adeeti V. Ullal, Emerald Hills, CA (US); Alexander G. Bruno, Cupertino, CA (US); Eugene Song, San Jose, CA (US); Gabriel A. Blanco, San Francisco, CA (US); James J. Dunne, San Francisco, CA (US); João Antunes, Campbell, CA (US); Karthik Jayaraman Raghuram, Foster City, CA (US); Po An Lin, San Jose, CA (US); Richard A. Fineman, Campbell, CA (US); William R. Powers, III, San Francisco, CA (US); Asif Khalak, Belmont, CA (US)

(21) Appl. No.: 18/205,472

(22) Filed: Jun. 2, 2023

Related U.S. Application Data
(60) Provisional application No. 63/349,084, filed on Jun. 4, 2022.

(51) Int. Cl. A61B 5/11; G16H 50/20; A61B 5/00

(52) U.S. Cl. CPC A61B 5/112; G16H 50/20; A61B 5/681; A61B 5/7267

(57) ABSTRACT

Enclosed are embodiments for estimating gait time events and GCT using a wrist-worn device. In some embodiments, a method comprises: obtaining, with at least one processor of a wrist-worn device, sensor data indicative of acceleration and rotation rate; and predicting, with the at least one processor, at least one gait event time based on a machine learning (ML) model with the acceleration and rotation rate as input to the ML model.

### CROSS-REFERENCE TO RELATED APPLICATION

This application claims priority to U.S. Provisional Patent Application No. 63/349,084, filed Jun. 4, 2022, the entire contents of which are incorporated herein by reference.

### TECHNICAL FIELD

This disclosure relates generally to health monitoring and fitness applications.

### BACKGROUND

Ground contact time (GCT) is the time spent while the foot is in contact with the ground (e.g., from foot strike, stance to toe-off) for each stride cycle while running. GCT is lower when running faster and when running at higher cadence. Lower GCT is generally associated with better running performance and running economy. Existing methods may estimate GCT using wearable sensors that attach to the center of mass of the runner (e.g., torso, waist) or foot to estimate GCT directly without producing timing predictions for the gait events, such as foot strike and toe-off events. Although wearing torso, waist or foot sensors may be acceptable for professional athletes in training, the average user may find such devices inconvenient and cumbersome. Also, many users may already own a smartwatch and may not want to purchase another wearable device that has limited application.

### SUMMARY

Embodiments are disclosed for estimating gait event times and GCT at the wrist.

In some embodiments, a method comprises: obtaining, with at least one processor of a wrist-worn device, sensor data indicative of acceleration and rotation rate; and predicting, with the at least one processor, at least one gait event time based on a machine learning (ML) model with the acceleration and rotation rate as input to the ML model.

In some embodiments, the method further comprises combining multiple predictions of GCT per running step.

In some embodiments, the at least one gait event time includes initial contact event time.

In some embodiments, the at least one gait event time includes toe-off event time. In some embodiments, the at least one gait event time includes GCT.

In some embodiments, prior to predicting, the sensor data is converted from a sensor reference coordinate frame to an inertial reference frame.

In some embodiments, the method further comprises averaging the predicted GCT over time.

In some embodiments, the method further comprises: determining GCT balance from the predicted GCT.

In some embodiments, the method further classifies predicted GCT as right foot GCT and/or left foot GCT.

In some embodiments, the method further comprises: determining GCT balance from predicted left and right GCT values

In some embodiments, the machine learning model is a neural network.

In some embodiments, the neural network is a long short-term memory (LSTM) neural network, or a mix of LSTM and feed forward neural networks.

In some embodiments, the neural network includes a single LSTM with three outputs that uses internal representations learned for gait events to predict GCT.

In some embodiments, the neural network includes an LSTM layer, encoding layers, a number of fully-connected layers or dense layer and an output layer.

In some embodiments, a system comprises: at least one processor; memory storing instructions that when executed by the at least one processor, cause the at least one processor to perform any of the preceding methods recited above.

In some embodiments, a non-transitory, computer-readable storage medium having stored thereon instructions that when executed by the at least one processor, causes the at least one processor to perform any of the preceding methods recited above.

Other embodiments can include an apparatus, computing device and non-transitory, computer-readable storage medium.

Particular embodiments described herein provide one or more of the following advantages. Gait event times and GCT can be determined by a single wrist worn device (e.g., smart watch), avoiding the inconvenience and cost of purchasing devices that are worn at the torso, waist or foot.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates running form metrics at the wrist, according to some embodiments.

FIG. 2 illustrates running form metrics from feet to wrist, according to some embodiments.

FIG. 3 is a block diagram of a machine learning (ML) network for estimating gait event times, according to some embodiments.

FIG. 4 is a block diagram of a ML network for estimating GCT, according to some embodiments.

FIG. 5 is flow diagram of a process of estimating gait event times and GCT at the wrist, according to some embodiments.

FIG. 6 is example system architecture implementing the features and operations described in reference to FIGS. 1-5.

### DETAILED DESCRIPTION

### Example System

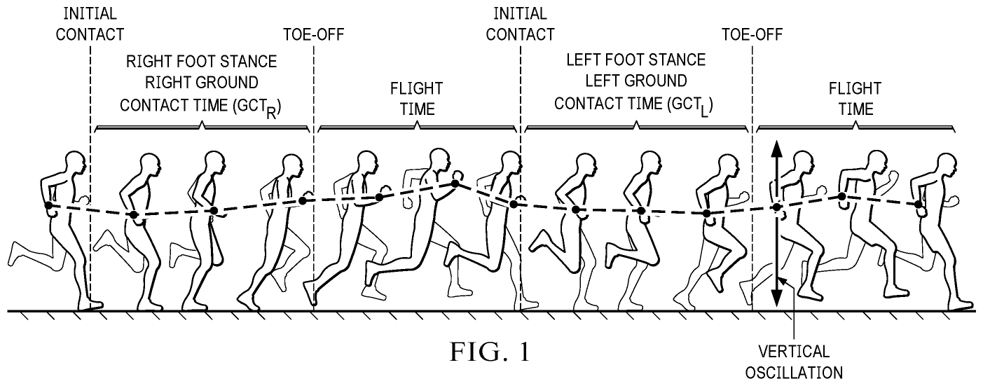

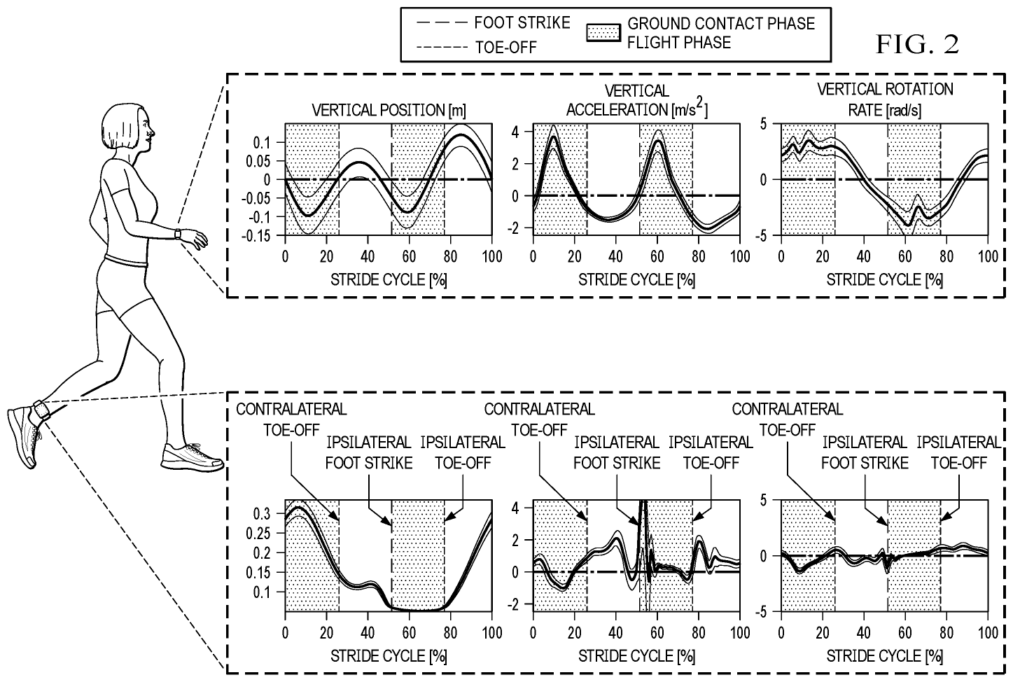

FIG. 1 illustrates running form metrics at the wrist, according to some embodiments. FIGS. 1 and 2 were adapted from Uchida, Thomas K. et. al., Biomechanics of Movement—The Science of Sports, Robotics, and Rehabilitation. MIT Press Ltd., United States, 2021.

In the example shown, a stride cycle is shown for computing total GCT. GCT for the right foot stance is measured from initial contact with the ground (right foot strike) to right foot toe off. The right foot stance GCT is followed by a first flight time (when both feet are off the ground). GCT for the left foot stance is measured from initial contact by the left foot (left foot strike) after the flight time to left foot toe-off, which is again followed by a second flight time. The addition of the right foot stance GCT and left foot stance GCT gives the total GCT for the stride cycle. As indicated by the dashed line in FIG. 1, vertical oscillation (bouncing) occurs at the runner's center of mass (CoM) during the stride cycle. The vertical oscillation (VO) can be used as a signal for calculating GCT.

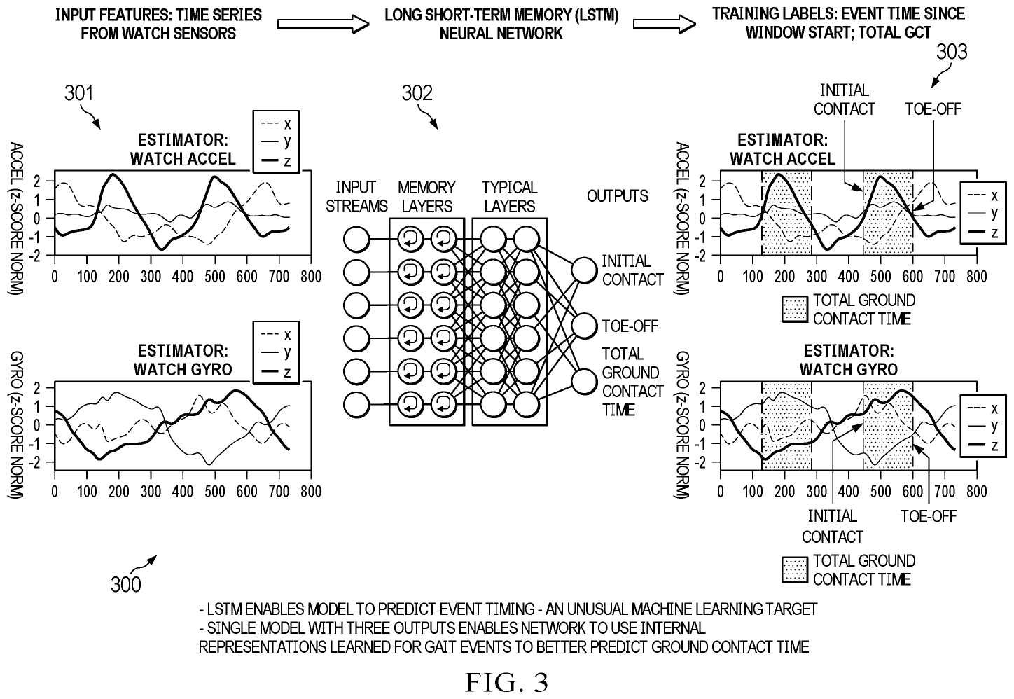

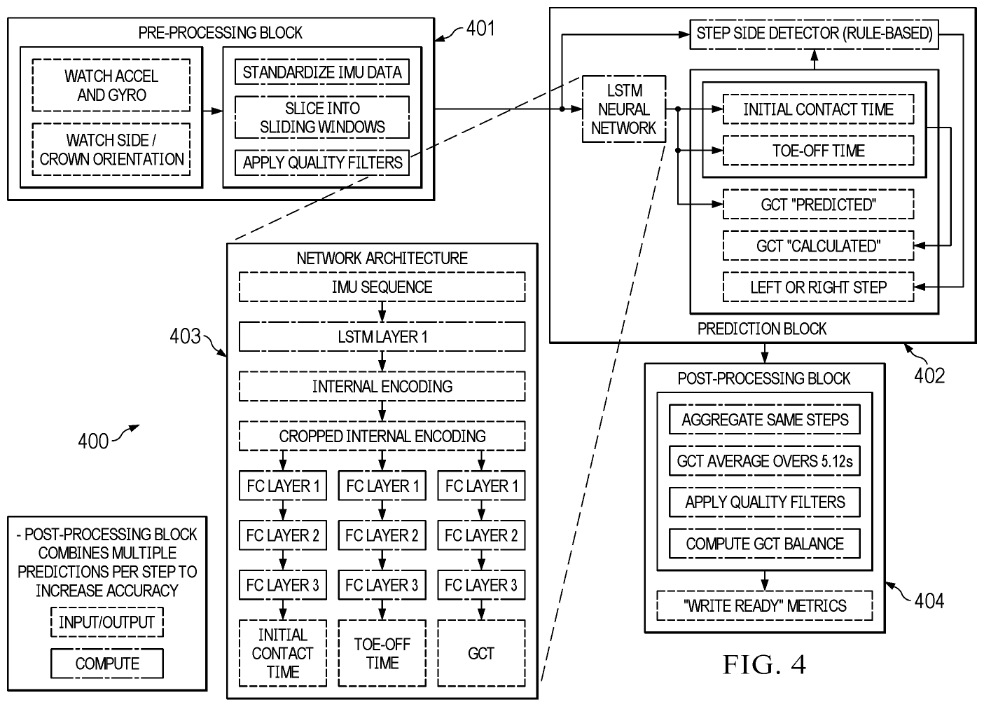

If the runner is wearing a sensor at the waist or torso (e.g., an accelerometer), VO can be measured closer to the CoM. If the runner is wearing a foot sensor, the runner's foot dynamics (foot strike, toe-off) can be measured directly, and the time between foot strike and toe-off can be used to determine GCT. If, however, the runner is wearing a single sensor on their wrist, vertical oscillation cannot be measured directly due to biomechanical linkage from feet to wrist. Although one could attempt to model the biomechanics with a biomechanical linkage model, such a model can be complex and will not account for population diversity as each runner may run in a different manner. An alternative solution is to infer lower body motion at the wrist using ML models, as described in reference to FIGS. 3 and 4.

FIG. 2 illustrates running dynamics at the feet and the wrist, according to some embodiments. The upper plots from left to right are vertical position (m), vertical acceleration (m/s2) and vertical rotation rate (rad/s) measured at the wrist, respectively, versus a percentage of stride cycle. The lower plots from left to right are also vertical position, vertical acceleration and vertical rotation rate, but measured at the feet rather than the wrist. The vertical dashed lines separate the different gait events: foot strike, toe-off, GTC phase and flight phase.

As can be observed from the plots, vertical position, acceleration and rotation rate dynamics at the feet are observable at the wrist as peaks, step periodicity (for acceleration) and stride periodicity (for rotation rate) with a phase shift from the gait event of interest. Thus, the vertical acceleration and rotation rate measured at the wrist are signals that can be used to infer gait event times using ML models.

FIG. 3 is a block diagram of ML network 300 for estimating gait event times, according to some embodiments. In the example shown, ML network 300 is a long short-term memory (LSTM) network. However, other types of ML networks suitable for estimating gait event times can also be used (e.g., other types of recurrent neural network (RNN)), including using two or more ML networks in series or parallel (e.g., stacked LSTMs), or combining LSTM and feed forward layers, etc.

Input layer 301 of LSTM network 300 receives windows of input streams (sequences of time-series sensor data), including 3-axis vertical acceleration data and 3-axis rotation rate data from inertial sensors (e.g., 3-axis accelerometer 3-axis gyro) embedded in a wrist-worn device, such as a smartwatch. Each window can include multiple steps. In some embodiments, LSTM network 300 includes memory and other typical LSTM layers (e.g., dense layer, softmax layer) and classification output layer 303. Classification output layer 303 predicts labels for various gait event times on each window, including but not limited to: initial contact time for the last step in the window (e.g., seconds from the start of the window), toe-off time of the last step in the window and total GCT of the last step in the window. In other embodiments, additional gait event times or other gait parameters can be estimated. The estimated GCTs for the right and the left foot, respectively, are shown as two shaded regions in the output plots of FIG. 3, which can be added together to give the total GCT for the stride cycle.

In some embodiments, LSTM network 300 is a single hidden layer model that uses internal representations learned for gait event times to infer gait event times and total GCT. In other embodiments, each gait event time is run separately through an LSTM network. In some embodiments, LSTM network 300 is a stacked LSTM network model with multiple LSTM layers.

In some embodiments, LSTM network 300 is trained in a supervised manner on a set of time-series data output collected at the wrist, including vertical acceleration and rotation rate. In some embodiments, the training uses a gradient descent process (e.g., stochastic gradient descent (SGD), Nesterov accelerated gradient, Adagrad, AdaDelta), or any other suitable optimizer (e.g., RNSProp, Adam), combined with back propagation through time to compute the data (e.g., gradients) needed during the optimization process to change each weight of LSTM network 300. In some embodiments, each weight is changed in proportion to the derivative of the error at the output layer 303 of LSTM network 300 with respect to corresponding weight. Activation functions for LSTM network 300 can include but are not limited to sigmoid functions or hyperbolic tangent functions.

FIG. 4 is a block diagram of ML network 400 for estimating GCT, according to some embodiments. In some embodiments, ML network 400 includes pre-processing block 401, prediction block 402, network block 403 and post-processing block 404. Pre-processing block 401 includes standardize IMU data generator 405. Prediction block 402 includes ML model 403 and step side detector 406.

Pre-processing block 401 receives watch device motion and orientation (e.g., a watch side/crown orientation for a smartwatch) and converts this data into standardized inertial measurement unit (IMU) coordinate frames, slices the data into sliding time windows used for training and apply quality factors to the data. Some examples of quality factors include but are not limited to: sufficient data in window, sufficient predictions in window, predictions in acceptable range, user in expected motion state and no unacceptable event (e.g., data skip, watch glance, etc.)

The output of pre-processing block 401 is provided as input into network 403 and step side detector 406. In some embodiments, network 403 is an artificial neural network comprising LSTM layer, internal encoding layers, fully-connected or dense layer (e.g., 3 layers) and output layer. Network 403 predicts/infers, for a stride cycle, initial contact time, toe-off time and GCT. In some embodiment, the predicted initial contact time and toe-off time are input into step side detector 406, together with the standardize IMU data, which predicts right or left foot for the contact and toe-off events.

Post-processing block 404 aggregates multiple GCT predictions output by prediction block 402 per step to increase the accuracy of the prediction and applies quality filters. In some embodiments, a sliding window (using a small time increment that is empirically determined) is applied to each window of data used to make a GCT prediction, resulting in multiple GCT predictions corresponding to the same physical step, which are aggregated over the stride cycle. The initial contact times of the GCT predictions are checked to ensure they are sufficiently close in time to provide confidence that the initial contact times correspond to the same physical step in the window. The GCT predictions are combined or aggregated to get an estimated GCT. In some embodiments, the aggregated GCT predictions are averaged to get an average estimated GCT.

In some embodiments, post-processing block 404 estimates GCT balance. GCT balance is a measure of how similar GCT is for a runner's left and right legs. In some embodiments, GCT balance is represented as a percentage split, where a 50/50 split is the theoretical optimum and indicates an equal GCT for both legs. In some embodiments, GCT balance is computed from a ratio of each of right leg GCT and left leg GCT over the total GCT to get the percentage of the total GCT time that the right leg and the left leg are in contact with the ground. A GCT balance value between of, for example, about 49% and 51% is considered fairly symmetrical. If the GCT balance is, for example, greater than 49% and 51% (an imbalance >2%) the asymmetry may affect the runner's performance and put the runner at risk of injury. In some embodiments, GCT balance thresholds are determined empirically using any suitable method.

Network architecture 403 includes various tunable parameters, including but not limited to: input window size, number of LSTM layers, size of the internal encoding window, number of hidden units in linear (fully-connected) layers, dropout probability and number of epochs for training.

### Example Processes

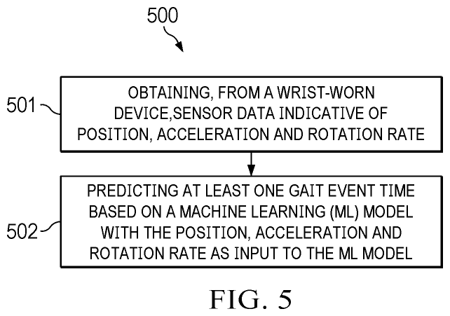

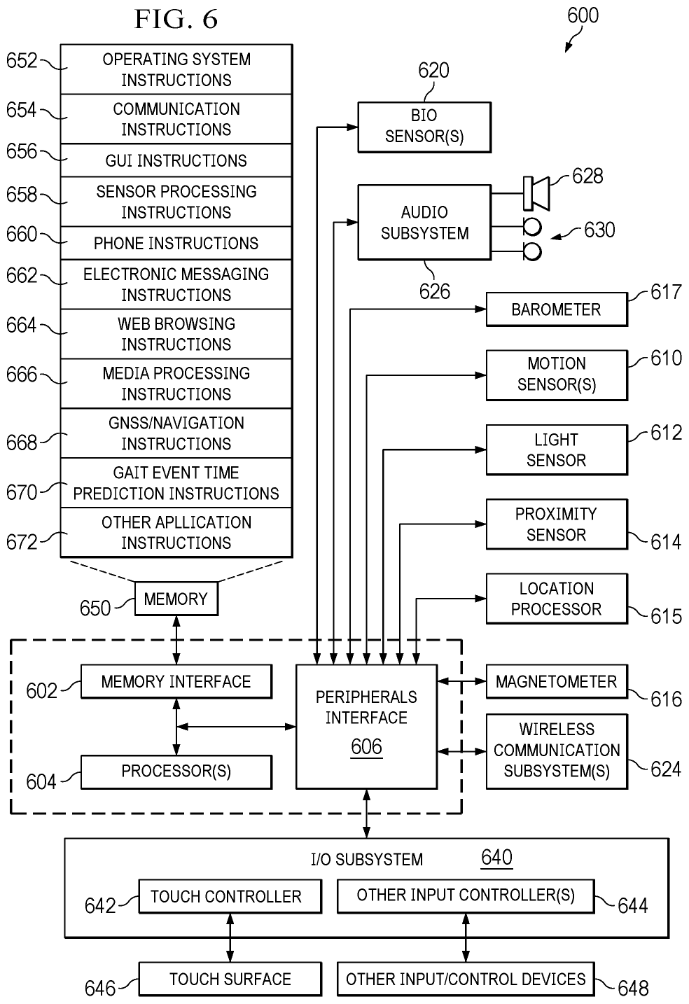

FIG. 5 is flow diagram of a process 500 of estimating gait event times and GCT at the wrist, according to some embodiments. Process 500 can be implemented by, for example, using system architecture 600 described in reference to FIG. 6.

Process 500 includes the steps of obtaining, from a wrist-worn device, sensor data indicative of acceleration and rotation rate (501), predicting at least one gait event time based on a machine learning (ML) model with the acceleration and rotation rate as input to the ML model (502). In some embodiments, the predicted gait time events include initial contact time, toe-off time and GCT. In some embodiments, the ML model is an LSTM network.

### Exemplary System Architectures

FIG. 6 illustrates example system architecture 600 implementing the features and operations described in reference to FIGS. 1-5. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits. System architecture 600 can be included in any suitable wearable device, including but not limited to: a smartphone, smartwatch, fitness band and the like.

Sensors, devices and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the mobile device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement. Barometer 617 can be configured to measure atmospheric pressure. Biosensors 620 can include a heart rate sensor, such as a photoplethysmography (PPG) sensor, electrocardiography (ECG) sensor, etc.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a Wi-Fi™ network and a Bluetooth™ network. In particular, the wireless communication subsystems 624 can include hosting protocols, such that the mobile device can be configured as a base station for other wireless devices.

Audio subsystem 626 can be coupled to a speaker 628 and a microphone 630 to facilitate voice-enabled functions, such as voice recognition, voice replication, digital recording and telephony functions. Audio subsystem 626 can be configured to receive voice commands from the user.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumb-wheel, infrared port and USB port. The one or more buttons (not shown) can include an up/down button for volume control of speaker 628 and/or microphone 640. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices, such as a sleep/wake tracking device. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and gait event time prediction instructions 670 that implement the features and processes described in reference to FIGS. 1-5. Memory 650 further includes application instructions 672 for various applications that use gait event times and/or GCT (e.g., health monitoring, fitness applications/framework (e.g., Apple's HealthKit™).

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor of a wrist-worn device, sensor data indicative of acceleration and rotation rate; and
predicting, with the at least one processor, at least one gait event time based on a machine learning (ML) model with the acceleration and rotation rate as input to the ML model.

2. The method of claim 1, further comprising combining multiple predictions of ground contact time (GCT) per running step.

3. The method of claim 2, further comprising:
averaging the multiple GCT predictions over time.

4. The method of claim 1, wherein the at least one gait event time includes initial contact event time.

5. The method of claim 1, wherein the at least one gait event time includes toe-off event time.

6. The method of claim 1, wherein the at least one gait event time includes ground contact time (GCT).

7. The method of claim 6, further comprising:
determining GCT balance from the predicted GCT.

8. The method of claim 7, further comprising determining GCT as right foot GCT or left foot GCT.

9. The method of claim 8, further comprising determining GCT balance from the determined left foot GCT or right foot GCT.

10. The method of claim 1, further comprising:
prior to predicting, converting the sensor data from a sensor reference coordinate frame to an inertial reference frame

11. The method of claim 1, wherein the machine learning model is a neural network.

12. The method of claim 1, wherein the neural network is a long short-term memory (LSTM) neural network.

13. The method of claim 11, wherein the neural network includes a single LSTM with three outputs that uses internal representations learned for gait events to predict GCT.

14. The method of claim 13, wherein the LSTM neural network includes an LSTM layer, encoding layers, a number of full-connected layers or dense layer and an output layer.

15. A system comprising:
at least one processor;
memory storing instructions that when executed by the at least one processor, cause the at least one processor to perform operations comprising:
obtaining data indicative of acceleration and rotation rate; and
predicting at least one gait event time based on a machine learning (ML) model with the acceleration and rotation rate as input to the ML model.

16. The system of claim 15, further comprising combining multiple predictions of ground contact time (GCT) per running step.

17. The system of claim 16, further comprising:
averaging the multiple GCT predictions over time.

18. The system of claim 15, wherein the at least one gait event time includes initial contact event time.

19. The system of claim 15, wherein the at least one gait event time includes toe-off event time.

20. The system of claim 15, wherein the at least one gait event time includes ground contact time (GCT).
